(19) United States
(12) Patent Application Publication
(10) Pub. No.: US 2005/0113449 A1
(43) Pub. Date: May 26, 2005

(54) Enhanced efficacy of omega-3 fatty acid therapy in the treatment of psychiatric disorders and other indications

(76) Inventor: Perry F. Renshaw, Bedford, MA (US)

Correspondence Address: CLARK & ELBING LLP, 101 FEDERAL STREET, BOSTON, MA, 02110, US

(21) Appl. No.: 10/961,288

(22) Filed: Oct. 8, 2004

Related U.S. Application Data
(60) Provisional application No. 60/509,622, filed on Oct. 8, 2003.

(51) Int. Cl. A61K 31/202

(52) U.S. Cl. 514/560

(57) ABSTRACT

The invention features an improved method for treating psychiatric disorders, such as mood disorders, attention deficit hyperactivity disorder (ADHD), anxiety disorders, obsessive-compulsive disorder (OCD), post-traumatic stress disorder (PTSD), phobias, substance abuse or dependency, and psychotic disorders, and other conditions, e.g., cardiovascular disease and cancer, using omega-3 fatty acids. The invention further provides methods of enhancing neurodevelopment and delaying premature pregnancy using omega-3 fatty acids. In this improved method, omega-3 fatty acids are delivered via a hydrophobic carrier, and this method of delivery reduces the time of onset of therapeutic effect. Examples of omega-3 fatty acids include eicosapentaenoic acid, docosahexaenoic acid, and α-linolenic acid.

### CROSS-REFERENCE TO RELATED APPLICATIONS

This application claims benefit of U.S. Provisional Application No. 60/509,622, filed Oct. 8, 2003, hereby incorporated by reference.

### BACKGROUND OF THE INVENTION

The invention relates to the fields of pharmaceutical formulations and treatment of psychiatric disorders and other indications.

Psychiatric disorders, such as depression and schizophrenia, affect millions of people worldwide. Medications are available to treat such disorders, but these medications typically have numerous potential side effects and are not effective for all individuals. In addition, medications administered to treat various psychiatric disorders often have a delayed onset of efficacy, e.g., from days to weeks. During this period, the patient still suffers from the symptoms of a particular disorder.

In addition, the latency period for achieving a beneficial effect in other indications, such as cardiovascular disease and cancer, may also be significant.

There is thus a need for improved methods of administering compounds that reduce the time of onset of effect.

### SUMMARY OF THE INVENTION

The invention features improved methods for treating psychiatric disorders, cardiovascular disease, cancer, dysmenorrhea, infertility, preeclampsia, postpartum depression, menopausal discomfort, osteoporosis, thrombosis, inflammation, hyperlipidemia, hypertension, rheumatoid arthritis, hyperglyceridemia, and gestational diabetes using omega-3 fatty acids. The invention also features improved methods for enhancing neurodevelopment and delaying premature pregnancy using omega-3 fatty acids. In these improved methods, omega-3 fatty acids are delivered via a hydrophobic carrier as described herein, and this method of delivery reduces the time of onset of therapeutic or other beneficial effect. For example, an effect may be achieved in less than 3, 7, 10, 14, 17, 21, 24, or 28 days. The onset of efficacy may also be at least 2, 3, 4, 5, or even 10 times faster than when omega-3 fatty acids are administered other than via a hydrophobic carrier, e.g., via a capsule. Examples of omega-3 fatty acids include eicosapentaenoic acid, docosahexaenoic acid, and α-linolenic acid. Exemplary methods of administration are described herein.

Exemplary psychiatric disorders that may be treated using the compositions described herein include mood disorders (e.g., depression, bipolar disorder, dysthymia, and cyclothymia), attention deficit hyperactivity disorder (ADHD), anxiety disorders (e.g., generalized anxiety disorder and panic disorder), obsessive-compulsive disorder (OCD), post-traumatic stress disorder (PTSD), phobias, substance abuse or dependency (e.g., from alcohol, stimulants, opiates, cocaine, amphetamines, methamphetamine, and methylphenidate), and psychotic disorders (e.g., schizoaffective disorder and schizophrenia).

Formulations of the invention include an omega-3 fatty acid, for example, as at least 1, 5, 10, 20, 30, 40, 50, 60, 70, 80, or 90% of the total hydrophobic carrier by weight.

Compositions of the invention may be administered concomitantly with other drugs, as described herein. In one embodiment, another drug is carried in the hydrophobic carrier, e.g., either dissolved in a lipid membrane or encapsulated in the interior of a liposome.

By “omega-3 fatty acid” is meant a fatty acid having an unsaturated bond three carbons from the omega carbon. This term encompasses the free acid, a salt, or an esterified form, e.g., a phospholipid. Omega-3 fatty acids may be mono- or polyunsaturated.

By “hydrophobic carrier” is meant a chemical or chemical aggregate capable of solubilizing an omega-3 fatty acid in a polar solvent, e.g., water. Hydrophobic carriers include inclusion complexes, micelles, microemulsions, emulsions, and liposomes.

By “treating” is meant the medical management of a patient with the intent that a prevention, cure, stabilization, or amelioration of the symptoms will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of the disorder; and supportive treatment, that is, treatment employed to supplement another specific therapy directed toward the improvement of the disorder. The term “treatment” also includes symptomatic treatment, that is, treatment directed toward constitutional symptoms of the disorder.

By “effective amount” is meant an amount of a pharmaceutical composition sufficient to produce the desired outcome of a method. For example, a therapeutically effective amount is an amount sufficient to produce a preventative, healing, curative, stabilizing, or ameliorative effect either in the treatment of a disorder or in the treatment of symptoms of a disorder.

By “reducing the time required for effect” is meant decreasing the latency time before the desired outcome is achieved, e.g., a preventative, healing, curative, stabilizing, or ameliorative effect either in the treatment of a disorder or in the treatment of symptoms of a disorder, relative to treatment with an omega-3 fatty acid in the absence of a hydrophobic carrier.

Unless otherwise stated, all psychiatric and substance abuse disorders are those described in Diagnostic and Statistical Manual of Mental Disorders, 4th ed., Text Revision, Washington, D.C.: American Psychiatric Association, 2000, hereby incorporated by reference.

Other features and advantages will be apparent from the following description and the claims.

### DETAILED DESCRIPTION OF THE INVENTION

Omega-3 fatty acids are believed to have efficacy in the treatment of psychiatric disorders, such as bipolar disorder and depression, and in other indications, as described herein. Typically, omega-3 fatty acids have administered through oral ingestion of the active ingredient, e.g., through fish oil capsules or by eating fatty fish. Animal models, however, indicate that the onset of efficacy using this method of delivery only occurs after an extended period of time, e.g., 30 days. These data indicate that oral delivery of omega-3 fatty acids is inefficient. Functional MRI measurements have also shown that omega-3 fatty acids increase membrane fluidity in the brain, and a method of administering omega-3 fatty acids that provides more rapid integration into brain cellular membranes should decrease the latency period of achieving the effect. Methods of administration of omega-3 fatty acids that allow a more rapid onset of beneficial effect are also desirably in other indications, as described herein.

The present invention features improved methods for treating psychiatric disorders and for other indications using omega-3 fatty acids. These improved methods involve the delivery of omega-3 fatty acids via a hydrophobic carrier, e.g., a liposome. Hydrophobic carriers may increase the rate in which omega-3 fatty acids provide an effect, e.g., by administration by parenteral routes or by protection from degradation or increase of absorption in the gastrointestinal tract.

Omega-3 Fatty Acids

Omega-3 fatty acids include eicosapentaenoic acid, docosahexaenoic acid, and α-linolenic acid. Omega-3 fatty acids may be administered as the free acid or in esterified form (e.g., as triglycerides or phospholipids). Omega-3 fatty acids may be obtained in pure form by synthesis or by culture of microalgae. Omega-3 fatty acids may also be administered in a mixture from a naturally occurring source, e.g., fish oil, flaxseed oil, soybeans, rapeseed oil, or microalgae. Omega-3 fatty acids are well tolerated in humans.

Omega-3 fatty acids may be used to treat any appropriate psychiatric disorder, e.g., those associated with a decrease in membrane fluidity. Psychiatric disorders include mood disorders (e.g., depression, bipolar disorder, and cyclothymia), attention deficit hyperactivity disorder (ADHD), anxiety disorders (e.g., generalized anxiety disorder and panic disorder), obsessive-compulsive disorder (OCD), post-traumatic stress disorder (PTSD), phobias, substance abuse or dependency (e.g., from alcohol, stimulants, opiates, cocaine, amphetamines, methamphetamine, and methylphenidate), and psychotic disorders (e.g., schizoaffective disorder, and schizophrenia). Exemplary psychiatric disorders are depression, dysthymia, cyclothymia, anxiety disorders (e.g., generalized anxiety disorder and panic disorder), obsessive-compulsive disorder (OCD), post-traumatic stress disorder (PTSD), phobias, and substance abuse or dependency (e.g., from alcohol, stimulants, opiates, cocaine, amphetamines, methamphetamine, and methylphenidate).

Omega-3 fatty acids may also be employed to enhance neurodevelopment, e.g., neurite growth. Methods for evaluating the enhancement of neurodevelopment are known in the art (e.g., Gibson, R. A. and M. Makrides Acta Paediatr, 1998, 87:1017-22, Fewtrell, M. S., et al., J Pediatr, 2004, 144:471-9, Fewtrell, M. S., et al., Pediatrics, 2002, 110:73-82, O'Connor, D. L., et al., Pediatrics, 2001, 108:359-71, Clandinin, M., et al., Pediatric Res, 2002, 51:187A-8A, Innis, S. M., et al., J Pediatr, 2002, 140:547-54, Clandinin, M. T., et al., Pediatr Res, 1997, 42:819-25, Uauy, R., et al., J Pediatr, 1994, 124:612-20, Werkman, S. H. and S. E. Carlson, Lipids, 1996, 31:91-7, Carlson, S. E., et al., Eur J Clin Nutr, 1994, 48 Suppl 2:S27-30, Vanderhoof, J., et al, J Pediatr Gastroenterol Nutr, 2000, 31:121-7, and Marszalek, J. R., et al., J Biol Chem, 2004, 279:23882-91). Exemplary methods for gauging neurodevelopment include the Bayley Mental Developmental Index (MDI), the Bayley Psychomotor Developmental Index (PDI), Knobloch, Passamanick and Sherrard's Developmental Screening Inventory, and the Fagan Test of Infant Intelligence. Enhancement can be measured, for example, relative to a control group, such as a group that did not receive the compounds of the invention.

Omega-3 fatty acids may also be employed to treat cardiovascular disease (CVD), including atherosclerosis, coronary artery disease, regression and decreased progression of coronary lesions, decrease in triglyceride blood levels, increase in HDL cholesterol, neutralization of LDL cholesterol, reduction in mortality from cardiac events, and decrease in ventricular tachycardia.

Omega-3 fatty acids may also be employed to treat cancer, including reducing the risk of developing cancer (Larsson, S. C., et al., Am J Clin Nutr, 2004, 79:935-45), treating cancer cachexia during radio and chemotherapy and increasing the rate of recovery (Heller, A. R., et al., Int J Cancer, 2004, 111:611-6), and treating cancer-associated wasting (Jatoi, A., et al., J Clin Oncol, 2004, 22:2469-76). Exemplary cancers include breast, colon, pancreatic, chronic myelogenous leukemic, and melanoma.

The methods of the invention also address a number of medical problems that exclusively or particularly effect women, e.g., dysmenorrhea, infertility (e.g., by increasing uterine blood flow), preeclampsia, postpartum depression, menopausal discomfort, and osteoporosis. Omega-3 fatty acids may also be employed to delay premature birth, e.g., by balancing eicosanoids involved in labor and improving placental blood flow.

Omega-3 fatty acids may also be used treat other indications, such as thrombosis, inflammation, hyperlipidemia, hypertension, rheumatoid arthritis, hyperglyceridemia, and gestational diabetes.

Hydrophobic Carriers

Omega-3 fatty acids are administered via a hydrophobic carrier. Any hydrophobic carrier capable of solubilizing an omega-3 fatty acid in a polar solution, e.g., aqueous, may be employed in the methods described herein. Hydrophobic carriers include inclusion complexes, dispersions (such as micelles, microemulsions, and emulsions), and liposomes. Exemplary hydrophobic carriers are inclusion complexes, micelles, and liposomes. These formulations are known in the art (Remington: The Science and Practice of Pharmacy 20^{th} ed., ed. Gennaro, Lippincott: Philadelphia, Pa. 2003). Omega-3 fatty acids may be incorporated into the hydrophobic carrier as the free acid, salt, or incorporated into another molecule, e.g., a triglyceride or phosphotriglyceride, as appropriate. Omega-3 fatty acids may be incorporated into hydrophobic carriers, for example as at least 1, 5, 10, 20, 30, 40, 50, 60, 70, 80, or 90% of the total carrier by weight. In addition, other compounds may be included either in the hydrophobic carrier or the solution, e.g., to stabilize the formulation.

Inclusion Complexes

An inclusion complex is the combination of a macrocyclic compound having an interior cavity into which a small molecule can enter. Exemplary macrocyclic compounds are those with a polar exterior and a non-polar interior, such as cyclodextrin. In this formulation, an omega-3 fatty acid will enter the cavity of an appropriate macrocycle and be sequestered from a polar solvent.

Dispersions

A dispersion is a two-phase system in which one phase is distributed as particles or droplets in the other phase. In the present case, omega-3 fatty acids may be distributed in a polar phase, e.g., water. Micelles are dispersions in which surface active agents, e.g., amphiphilic compounds, form spherical particles where the polar groups are oriented on the outside of the particle in contact with the polar phase and the nonpolar groups are oriented in the interior of the particle. Omega-3 fatty acids may be part of the surfactants making up the micelle or may be dissolved in the interior of a micelle. Microemulsions are liquid dispersions of a polar solvent and an omega-3 fatty acid to which are added a surfactant and cosurfactant to produce a homogeneous, transparent, and stable dispersion. Emulsions are two-phase systems in which one liquid is dispersed throughout another liquid in the form of small droplets. In the present invention, an oil-in-water-emulsion is typically employed. In this formulation, the omega-3 fatty acid is solubilized in the oil droplets dispersed in the aqueous phase.

Liposomes

Liposomes are lipid vesicles made of membrane-like lipid bilayers separated by aqueous layers. Liposomes have been widely used to encapsulate biologically active agents for use as drug carriers since water- or lipid-soluble substances may be entrapped within the aqueous layers or within the bilayers themselves. There are numerous variables that can be adjusted to optimize this drug delivery system. These include, the number of lipid layers, size, surface charge, lipid composition and the methods of preparation.

Liposomes have been utilized in numerous pharmaceutical applications, including injectable, inhalation, oral and topical formulations, and provide advantages such as controlled or sustained release, enhanced drug delivery, and reduced systemic side effects as a result of delivery localization. Liposomes can interact directly with cells in vivo.

Potential mechanisms for this interaction include endocytosis, adsorption to the cell surface (through either specific or non-specific interactions), fusion with the cell membrane, and transfer of liposomal lipids to or from cellular or subcellular membranes. In addition, liposomes may be targeted to specific tissues by the inclusion of binding moieties, e.g., proteins such as antibodies or carbohydrates, that are specific for a desired type of cell.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. In general, lipids, such as omega-3 fatty acids, or lipophilic substances are dissolved in an organic solvent. When the solvent is removed, such as under vacuum by rotary evaporation, the lipid residue forms a film on the wall of the container. An aqueous solution that may contain electrolytes or hydrophilic biologically active materials is then added to the film. Large MLVs are produced upon agitation. When smaller MLVs are desired, the larger vesicles may be subjected to sonication, sequential filtration through filters with decreasing pore size, or reduced by other forms of mechanical shearing. There are also techniques by which MLVs can be reduced both in size and in number of lamellae, for example, by pressurized extrusion.

Liposomes can also take the form of unilamellar vesicles, which are prepared by more extensive sonication of MLVs and consist of a single spherical lipid bilayer surrounding an aqueous solution. Unilamellar vesicles (“ULVs”) can be small, having diameters of 20 to 200 nm, or they may be larger, having diameters of 200 nm to 2 μm. Methods of making unilamellar vesicles are known in the art, see, for example, U.S. Pat. No. 4,089,801, Batzri, et al., Biochim Biophys Acta 298:1015-1019 (1973), Deamer, et al., Biochim Biophys Acta 443:629-634 (1976), U.S. Pat. No. 4,235,871, and U.S. Pat. No. 4,016,100. In addition to the MLVs and ULVs, liposomes can also be multivesicular, i.e., spherical while containing internal granular structures (Kim, et al., Biochim Biophys Acta 728:339-348 (1983)). The outer membrane is a lipid bilayer and the internal region contains small compartments separated by bilayer septum. Oligolamellar vesicles (“OLVs”) have a large center compartment surrounded by several peripheral lipid layers. Additional methods of manufacturing liposomes are described in Liposome Technology, 2^{nd} ed., ed. G. Gregoriadis, CRC Press Inc., Boca Raton, Fla. 1992 and U.S. Pat. Nos. 4,485,054, 4,761,288, 5,653,996, and 5,013,497. Other methods are known in the art.

Exemplary lipids that may be used in liposomes include phosphatidyl cholines, lysophosphatidyl cholines, phosphatidyl serines, phosphatidyl ethanolamines, and phosphatidyl inositols. Additional lipids are known in the art.

Administration

Hydrophobic carriers containing omega-3 fatty acids may be administered by any suitable route. Exemplary routes include oral, parenteral, intravenous, subcutaneous, intramuscular, intracranial, intraorbital, ophthalmic, intraventricular, intracapsular, intraspinal, intracisternal, intraperitoneal, intranasal, or aerosol administration. Preferably, liposomes are administered intravenously. Omega-3 fatty acids may be administered at a dose of 0.5-100 grams per day.

Combination with other Therapeutics

The omega-3 fatty acid compositions of the invention may be administered as a monotherapy or in combination with other compounds for the treatment of psychiatric disorders. Preferably, the compounds of the invention, may be administered in conjunction with lower doses of current treatments for these disorders, including stimulants and antidepressants. For example, the compositions of the invention may be administered as an adjunct to standard therapy for the treatment of psychiatric disorders. These other compounds may be administered separately and by a different route of administration than the omega-3 fatty acids. Alternatively, additional compounds may be administered via the hydrophobic carrier, e.g., in the interior of a liposome or dissolved in a lipid layer.

In one particular example, omega-3 fatty acids of the invention may be administered in combination with an antidepressant, anticonvulsant, antianxiety, antimanic, antipyschotic, antiobsessional, sedative-hypnotic, stimulant, or anti-hypertensive medication. Examples of these medications include, but are not limited to, the antianxiety medications, alprazolam, buspirone hydrochloride, chlordiazepoxide, chlordiazepoxide hydrochloride, clorazepate dipotassium, desipramine hydrochloride, diazepam, halazepam, hydroxyzine hydrochloride, hydroxyzine pamoate, lorazepam, meprobamate, oxazepam, prazepam, prochlorperazine maleate, prochlorperazine, prochlorperazine edisylate, and trimipramine maleate; the anticonvulsants, amobarbital, amobarbital sodium, carbamazepine, chlordiazepoxide, chlordiazepoxide hydrochloride, clorazepate dipotassium, diazepam, divalproex sodium, ethosuximide, ethotoin, gabapentin, lamotrigine, magnesium sulfate, mephenyloin, mephobarbital, methsuximide, paramethadione, pentobarbital sodium, phenacemide, phenobarbital, phenobarbital sodium, phensuximide, phenyloin, phenyloin sodium, primidone, secobarbital sodium, trimethadione, valproic acid, and clonazepam; the antidepressants, amitriptyline hydrochloride, amoxapine, bupropion hydrochloride, clomipramine hydrochloride, desipramine hydrochloride, doxepin hydrochloride, fluoxetine, fluvoxamine, imipramine hydrochloride, imipramine pamoate, isocarboxazid, lamotrigine, maprotoline hydrochloride, nortriptyline hydrochloride, paroxetine hydrochloride, phenelzine sulfate, protriptyline hydrochloride, sertraline hydrochloride, tranylcypromine sulfate, trazodone hydrochloride, trimipramine maleate, and venlafaxine hydrochloride; the antimanic medications, lithium carbonate and lithium citrate; the antiobsessional medications, fluvoxamine, and clomipramine hydrochloride; the antipsychotic medications, acetophenazine maleate, chlorpromazine hydrochloride, chlorprothixene, chlorprothixene hydrochloride, clozapine, fluphenazine decanoate, fluphenazine enathrate, fluphenazine hydrochloride, haloperidol decanoate, haloperidol, haloperidol lactate, lithium carbonate, lithium citrate, loxapine hydrochloride, loxapine succinate, mesoridazine besylate, molindone hydrochloride, perphenazine, pimozide, prochlorperazine maleate, prochlorperazine, prochlorperazine edisylate, promazine hydrochloride, risperidone, thioridazine, thioridazine hydrochloride, thiothixene, thiothixene hydrochloride, and trifluoperzine hydrochloride; the sedative-hypnotic medications, amobarbital, amobarbital sodium, aprobarbital, butabarbital, chloral hydrate, chlordiazepoxide, chlordiazepoxide hydrochloride, clorazepate dipotassium, diazepam, diphenhydramine, estazolam, ethchlorvynol, flurazepam hydrochloride, glutethimide, hydroxyzine hydrochloride, hydroxyzine pamoate, lorazepam, methotrimeprazine hydrochloride, midazolam hydrochloride, oxazepam, pentobarbital sodium, phenobarbital, phenobarbital sodium, quazepam, secobarbital sodium, temazepam, triazolam, and zolpidem tartrate; the stimulants, dextroamphetamine sulfate, methamphetamine hydrochloride, methylphenidate hydrochloride, and pemoline; and the anti-hypertensive, clonidine.

Omega-3 fatty acids may also be administered with compounds for treating cardiovascular disease, cancer, dysmenorrhea, infertility, preeclampsia, postpartum depression, menopausal discomfort, osteoporosis, thrombosis, inflammation, hyperlipidemia, hypertension, rheumatoid arthritis, hyperglyceridemia, or gestational diabetes, enhancing neurodevelopment, or delaying premature birth. Examples of such compounds are cholesterol lowering drugs, antithrombotics, antiinflammatories, antihypertensives, analgesics, and chemotherapeutic agents.

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

All publications, patents, and patent applications mentioned in this specification are herein incorporated by reference to the same extent as if each individual publication, patent, or patent application was specifically and individually to be incorporated by reference.

Other embodiments are in the claims.

1. A method of treating a psychiatric disorder comprising administering to a patient a therapeutically effective amount of an omega-3 fatty acid associated with a hydrophobic carrier.

2. The method of claim 1, wherein said psychiatric disorder is a mood disorder, an anxiety disorder, obsessive-compulsive disorder, post-traumatic stress disorder, a phobia, substance abuse, or substance dependency.

3. The method of claim 2, wherein said mood disorder is depression, dysthymia, or cyclothymia.

4. The method of claim 2, wherein said mood disorder is bipolar disorder.

5. The method of claim 2, wherein said anxiety disorder is generalized anxiety disorder or panic disorder.

6. The method of claim 1, wherein said psychiatric disorder is attention deficit hyperactivity disorder (ADHD) or a psychotic disorder.

7. The method of claim 6, wherein said psychotic disorder is schizophrenia or schizoaffective disorder.

8. The method of claim 1, wherein said omega-3 fatty acid is docosahexaenoic acid, or α-linolenic acid.

9. The method of claim 1, wherein said omega-3 fatty acid is eicosapentaenoic acid.

10. The method of claim 1, wherein said administering occurs parenterally.

11. The method of claim 1, wherein said hydrophobic carrier is selected from the group consisting of an inclusion complex, a micelle, and a liposome.

12. The method of claim 1, wherein said hydrophobic carrier is an emulsion or a microemulsion.

13. The method of claim 1, wherein said administering reduces the time required for therapeutic effect.

14. The method of claim 1, wherein a therapeutic effect occurs in less than 3, 7, 10, 14, 17, 21, 24, or 28 days.

15. A method of treating cardiovascular disease, cancer, dysmenorrhea, infertility, preeclampsia, postpartum depression, menopausal discomfort, osteoporosis, thrombosis, inflammation, hyperlipidemia, hypertension, rheumatoid arthritis, hyperglyceridemia, or gestational diabetes comprising administering to a patient a therapeutically effective amount of an omega-3 fatty acid associated with a hydrophobic carrier.

16. The method of claim 15, wherein said omega-3 fatty acid is eicosapentaenoic acid, docosahexaenoic acid, or α-linolenic acid.

17. The method of claim 15, wherein said administering occurs parenterally.

18. The method of claim 15, wherein said hydrophobic carrier is selected from the group consisting of an inclusion complex, a micelle, and a liposome.

19. The method of claim 15, wherein said hydrophobic carrier is an emulsion or a microemulsion.

20. The method of claim 15, wherein said administering reduces the time required for therapeutic effect.

21. The method of claim 15, wherein a therapeutic effect occurs in less than 3, 7, 10, 14, 17, 21, 24, or 28 days.

22. A method of enhancing neurodevelopment in a mammal, comprising administering to a patient an effective amount of an omega-3 fatty acid associated with a hydrophobic carrier.

23. The method of claim 22, wherein said omega-3 fatty acid is eicosapentaenoic acid, docosahexaenoic acid, or α-linolenic acid.

24. The method of claim 22, wherein said administering occurs parenterally.

25. The method of claim 22, wherein said hydrophobic carrier is selected from the group consisting of an inclusion complex, a micelle, and a liposome.

26. The method of claim 22, wherein said hydrophobic carrier is an emulsion or a microemulsion.

27. The method of claim 22, wherein said administering reduces the time required for enhancing neurodevelopment.

28. A method of delaying premature birth in a mammal, comprising administering to a patient an effective amount of an omega-3 fatty acid associated with a hydrophobic carrier.

29. The method of claim 28, wherein said omega-3 fatty acid is eicosapentaenoic acid, docosahexaenoic acid, or α-linolenic acid.

30. The method of claim 28, wherein said administering occurs parenterally.

31. The method of claim 28, wherein said hydrophobic carrier is selected from the group consisting of an inclusion complex, a micelle, and a liposome.

32. The method of claim 28, wherein said hydrophobic carrier is an emulsion or a microemulsion.

33. The method of claim 28, wherein said administering reduces the time required for effect.
